(19) United States
(12) Patent Application Publication
(10) Pub. No.: US 2011/0236316 A1
(43) Pub. Date: Sep. 29, 2011

(54) PEPTIDE TARGETING IMAGING AGENTS AND METHODS OF USE THEREOF

(75) Inventors: Zheng-Rong Lu, Beachwood, OH (US); Furong Ye, Salt Lake City, UT (US)

(73) Assignee: University of Utah Research Foundation

(21) Appl. No.: 12/919,931

(22) PCT Filed: Feb. 27, 2009
(86) PCT No.: PCT/US09/35363

§ 371 Date: Jun. 10, 2011

Related U.S. Application Data
(60) Provisional application No. 61/032,489, filed on Feb. 29, 2008.

(51) Int. Cl. A61K 49/00; C07K 7/64; C07K 7/06; A61P 43/00; C12Q 1/02

(52) U.S. Cl. 424/9.2; 530/317; 530/330; 424/9.1; 435/29

(57) ABSTRACT

Described herein are peptide targeting imaging agents. The peptides are covalently attached to the imaging agent and have a specific peptide sequence that enables the imaging agent to accumulate specifically in tumor tissues. Additionally, the imaging agents are readily excreted by the subject within a short period of time after administration to the subject. Methods for using the imaging agents are also described herein.

### CROSS REFERENCE TO RELATED APPLICATIONS

This application claims priority upon U.S. provisional application Ser. No. 61/032,489, filed Feb. 29, 2008. This application is hereby incorporated by reference in its entirety for all of its teachings.

### ACKNOWLEDGEMENTS

The research leading to this invention was funded in part by the National Institutes of Health, Grant No. CA097465. The U.S. Government may have certain rights in this invention.

### CROSS REFERENCE TO SEQUENCE LISTING

Peptides described herein are referred to by a sequence identifier number (SEQ ID NO). The SEQ ID NO corresponds numerically to the sequence identifiers <400>1, <400>2, etc. The Sequence Listing, in written computer readable format (CFR), is incorporated by reference in its entirety.

### BACKGROUND

Probes and contrast agents are recognized as potential agents useful in cancer detection and diagnosis. For example, macromolecular MRI contrast agents have been investigated extensively. However, probes and contrast agents currently in use are much more difficult to excrete, which is mainly due to their excessive molecular mass. In order for a probe or contrast agent to be effective, it should exhibit specific targeting to disease tissues, yet small enough to be excreted from blood stream after examination. If the probe or contrast agent accumulates in bone and tissue due to poor clearance from the subject, toxicity and adverse side-effects can occur. Described herein are imaging agents that address these needs.

### SUMMARY

Described herein are peptide targeting imaging agents. The peptides are covalently attached to the imaging agent and have a specific peptide sequence that enables the imaging agent to accumulate specifically in tumor tissues. Additionally, the imaging agents are readily excreted by the subject within a short period of time after administration to the subject. Methods for using the imaging agents are also described herein. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the chemical structures of SEQ ID NO 1, SEQ ID NO 2, or covalently attached to an imaging agent (IA).

FIG. 2 shows the chemical structure of CLT1-(Gd-DTPA).

FIG. 3 shows the relaxivity calculation plot for T1 (A) and T2 (B) relaxivities.

FIG. 4 shows the fluorescence images of tumor tissues of mice bearing MDA-MB 231 xenografts before and 2 hours post-injection (A before injection; B, C 2 hours post injection).

FIG. 5A shows MR images of mice bearing MDA-MB 231 xenografts before contrast and at 1, 5, 15, 30 and 60 minutes post-injection of (A) CLT1-(Gd-DTPA) (0.05 mmol/kg) and (B) Omniscan (0.1 mmol/kg). FIG. 5B shows 2D T1-weighted SE images of mice bearing MDA-MB 231 xenografts before contrast and at 1, 5, 15, 30 and 60 minutes post-injection of (A) CLT1-(Gd-DTPA) (0.05 mmol/kg) and (B) Omniscan (0.1 mmol/kg).

FIG. 6A shows MR images of mice bearing U87 xenografts before contrast and at 1, 5, 15, 30 and 60 minutes post-injection of (A) CLT1-(Gd-DTPA) (0.05 mmol/kg) and (B) Omniscan (0.1 mmol/kg). FIG. 6B shows 2D T1-weighted SE images of mice bearing U87 xenografts before contrast and at 1, 5, 15, 30 and 60 minutes post-injection of (A) CLT1-(Gd-DTPA) (0.05 mmol/kg) and (B) Omniscan (0.1 mmol/kg).

FIG. 7 shows T₁-weighted 2D spin-echo images of mice bearing HT-29 xenografts before contrast and at 10, 30 and 60 minutes post-injection of CLT1-(Gd-DTPA), Omniscan® and competitive study.

FIG. 8 shows plots of CNR versus time in tumor periphery (A) and inner area (B) before contrast and at 10, 30 and 60 minutes post-injection of CLT1-(Gd-DTPA), Omniscan® and competitive study.

FIG. 9 shows staining of fibronectin in HT-29 tumor tissues with hemoxylin (9A) or without hemoxylin (9B). Arrows point to the stained fibronectin (brown color) in the EES space, and blue in 9A indicates the nucleus was stained with hemoxylin (magnification 40×).

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

The term “peptide” may be used to refer to a natural or synthetic molecule comprising two or more amino acids linked by the carboxyl group of one amino acid to the alpha amino group of another. The peptide is not limited by length, and thus “peptide” can include polypeptides and proteins.

The term “degenerate variant” refers to an amino acid or peptide sequence having conservative amino acid substitutions, amino acids added to the C-terminus of a peptide, or a peptide having 60%, 70%, 80%, 90%, or 95% homology to SEQ ID NO 1, SEQ ID NO 2, or SEQ ID NO 3 as described herein.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

Described herein are imaging agents that specifically accumulate in tumor tissues. In one aspect, the imaging agent has a cyclic peptide covalently attached to an imaging agent, wherein the peptide includes the sequence CGLIIQKNEC SEQ ID NO 1 (also referred to herein as “CLT1”), CNAGESSKNC SEQ ID NO 2 (also referred to herein as “CLT2”), or a linear peptide comprising the sequence CREKA SEQ ID NO 3.

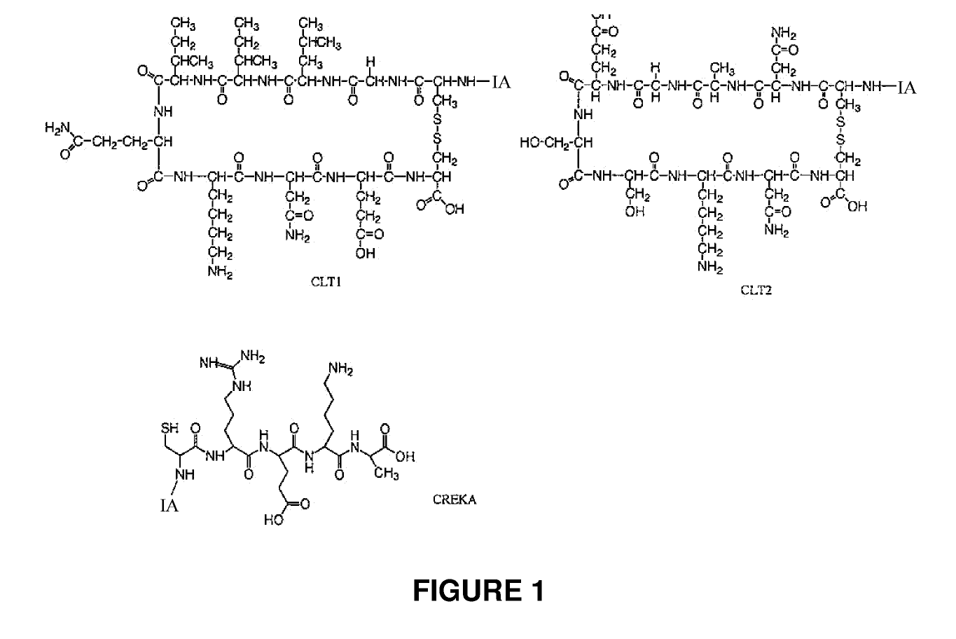

The structures of the peptides are shown in FIG. 1. Referring to FIG. 1, the peptide is covalently attached to an imaging agent (IA). In general, the peptide is covalently attached to the imaging agent by an amide bond. The synthesis and characterization of SEQ ID NO 1 and SEQ ID NO 2 is described in Pilch et al. “Peptides Selected for Binding to Clotted Plasma Accumulate in Tumor Stroma and Wounds” Proc. Nat. Acad. Sci., vol. 103, no. 8, 2800-2804 (2006). The synthesis and characterization of SEQ ID NO 3 is described in Samberg et al. “Biomimetic amplification of nanoparticles homing to tumors” Proc. Nat. Acad. Sci., vol. 104, no. 3, 932-936 (2006).

The covalent attachment of the peptide to an imaging agent can be performed using techniques known in the art. For example, the peptide can be reacted with an imaging agent, where the imaging agent possesses a group that can react with an amine group on the peptide. Examples of such groups include esters, carboxylic acids, anhydrides, epoxides, and the like. Referring to FIG. 1, SEQ ID NO 1 and SEQ ID NO 2 are cyclic disulfide complexes where an amino group pendant to the ring is covalently attached to the imaging agent. In other aspects, the imaging agent possesses a group that can also be reacted with a carboxylic group on the peptide.

A variety of different imaging agents can be used herein. The term “imaging agent” is defined herein as any agent or compound that increases or enhances the ability of neoplastic or antherosclerotic tissues to be imaged or viewed using techniques known in the art when compared to visualizing the cells or tissue without the imaging agent. Examples of imaging agents include, but are not limited to, an optical dye, a MRI contrast agent, a PET probe, a SPECT probe, a CT contrast agent, or an ultrasound contrast agent.

In certain aspects, the imaging agent includes a chelating agent and a metal ion. The chelating agent generally possesses one or more groups capable of forming a covalent bond with the peptide. A number of different chelating agents known in the art can be used herein. In one aspect, the chelating agent comprises an acyclic or cyclic compound comprising at least one heteroatom (e.g., oxygen, nitrogen, sulfur, phosphorous) that has lone-pair electrons capable of coordinating with the imaging agent. An example of an acyclic chelating agent includes ethylenediamine. Examples of cyclic chelating agents include diethylenetriaminepentaacetate (DTPA) or its derivatives, 1,4,7,10-tetraazadodecanetetraacetate (DOTA) and its derivatives, 1,4,7,10-tetraazadodecane-1,4,7-triacetate (DO3A) and its derivatives, ethylenediaminetetraacetate (EDTA) and its derivatives, 1,4,7,10-tetraazacyclotridecanetetraacetic acid (TRITA) and its derivatives, 1,4,8,11-tetraazacyclotetradecane-1,4,8,11-tetraacetic acid (TETA) and its derivatives, 1,4,7,10-tetraazadodecanetetramethylacetate (DOTMA) and its derivatives, 1,4,7,10-tetraazadodecane-1,4,7-trimethylacetate (DO3MA) and its derivatives, N,N′,N″,N′″-tetraphosphonatomethyl-1,4,7,10-tetraazacyclododecane (DOTP) and its derivatives, 1,4,7,10-tetraazacyclododecane-1,4,7,10-tetrakis(methylene methylphosphonic acid) (DOTMP) and its derivatives, 1,4,7,10-tetraazacyclododecane-1,4,7,10-tetrakis(methylene phenylphosphonic acid) (DOTPP) and its derivatives, or N,N′-ethylenedi-L-cysteine or its derivatives. The term “derivative” is defined herein as the corresponding salt and ester thereof of the chelating agent.

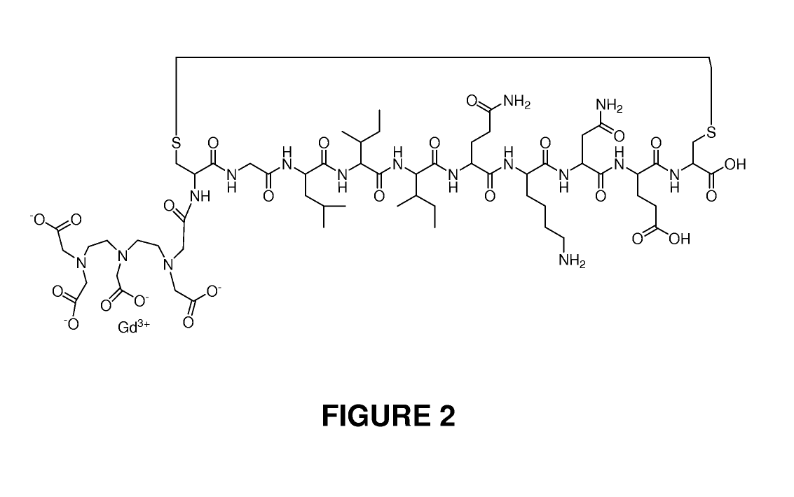

The selection of the metal ion can vary depending upon the detection technique (e.g., MRI, PET, etc.). In one aspect, metal ions useful in magnetic resonance imaging include Gd⁺³, Eu⁺³, Tm⁺³, Dy⁺³, Yb⁺³, Mn⁺², or Fe⁺³ ions. In another aspect, ions useful in PET and SPECT imaging include ⁵⁵Co, ⁶⁴Cu, ⁶⁷Cu, ⁴⁷Sc, ⁶⁶Ga, ⁶⁸Ga, ⁹⁰Y, ⁹⁷Ru, ^{99m}Tc, ¹¹¹h, ¹⁰⁹Pd, ¹⁵³Sm, ¹⁷⁷Lu, ¹⁸⁶Re, ¹⁸⁸Re. In another aspect, the imaging agent comprises an MRI agent, wherein the MRI agent comprises a chelating agent and a metal ion comprising Gd⁺³, Eu⁺³, Tm⁺³, Dy⁺³, Yb⁺³, Mn⁺², or Fe⁺³ ions. In a further aspect, the imaging agent comprises Gd⁺³ chelated to diethylenetriaminepentaacetate (DTPA) and the peptide is SEQ ID NO 1, which is depicted in FIG. 2.

Any of the compounds described herein can exist or be converted to the pharmaceutically acceptable salt. The salts can be prepared by treating the free acid with an appropriate amount of a chemically or pharmaceutically acceptable base. Representative chemically or pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. The molar ratio of the compound to base used is chosen to provide the ratio desired for any particular salts. For preparing, for example, the ammonium salts of the free acid starting material, the starting material can be treated with approximately one equivalent of base to yield a salt.

If the compounds possess carboxylic acid groups, these groups can be converted to pharmaceutically acceptable esters using techniques known in the art. Alternatively, if an ester is present on the compound, the ester can be converted to a pharmaceutically acceptable ester using transesterification techniques.

Pharmaceutical compositions composed of the imaging agents described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity-enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol.

The imaging agents described herein can be used to image a tissue in a subject. In one aspect, the method comprises (1) administering to the subject an imaging agent described herein, and (2) detecting the imaging agent. In one aspect, the imaging agents described herein can be used to image cancer cells and tumors. Examples of different types of cancers include, but are not limited to, breast cancer, liver cancer, stomach cancer, colon cancer, pancreatic cancer, ovarian cancer, lung cancer, kidney cancer, brain cancer, prostate cancer, testicular cancer, glioblastoma, sarcoma, bone cancer, head-and-neck cancers, and skin cancer. The agents can also be used in detecting wounds and atherosclerotic plaques. Not wishing to be bound by theory, it is believed that the peptides used herein bind to clotted plasma protein in diseased tissues, but not to normal plasma protein in healthy tissues.

In one aspect, the imaging agents described herein are useful in imaging fibrin-fibronectin complexes present in a subject. For example, MR molecular imaging of fibrin-fibronectin complexes in tumor tissue using the imaging agents described herein can be used to characterize tumor angiogenesis. It has been reported that the presence of the fibrin in tumor meshwork is associated with increased microvessel permeability in neoplastic tissues (Dvorak, H. F., Senger, D. R., Dvorak, A. M., Harvey, V. S., McDonagh, J. (1985) Regulation of extravascular coagulation by microvascular permeability. Science. 227, 1059-61) and fibronectin in tumor stroma is also associated with tumor angiogenesis (Neri, D., Carnemolla, B., Nissim, A., Leprini, A., Querzè, G., Balza, E., Pini, A., Tarli, L., Halin, C., Neri, P., Zardi, L., Winter, G. (1997) Targeting by affinity-matured recombinant antibody fragments of an angiogenesis associated fibronectin isoform. Nat. Biotechnol. 15, 1271-5). The imaging agents described herein can be used to detect the presence of fibrin-fibronectin complexes in the angiogenic tumor tissues. The correlation of fibrin-fibronectin complexes to tumor angiogenesis may provide an effective method for tumor angiogenesis imaging with MRI. Accurate assessment of tumor angiogenesis is critical for tumor grading, assessment of tumor response to anticancer therapies, particularly antiangiogenesis therapies, and patient management.

Fibrin-fibronectin complexes are also present in the plasma clots of wounds and other pathologic tissues with leaky blood vessels such as atherosclerotic plaques. Fibrin and fibronectin play a prominent role in hemostasis and wound healing. Fibrinogen is activated to form insoluble fibrin clot following vascular injury. The clot also serves as a provisional matrix for adhesion and migration of cells or proteins including fibronectin, which is incorporated into the fibrin clot upon fibrin polymerization. Thus, the imaging agents described herein can be used to image vascular integrity and assess wound healing, anthrosclerosis and tumor response to antiangiogenesis therapies.

Techniques known in the art for detecting the imaging agent once incorporated into the cells or tissue are known in the art. For example, magnetic resonance imaging (MRI), positron emission tomography (PET), single photon emission computed tomography (SPECT) and optical cameras can be used to detect the imaging agent. Additionally, the imaging agents described herein can be indispensable tools in a variety of other medical procedures, including, but not limited to, angiography, plethysmography, lymphography, mammography, cancer diagnosis, and functional and dynamic MRI.

The imaging agents described herein are useful in evaluating the performance of bioactive agents (e.g., drugs). In one aspect, a method for evaluating the ability of a bioactive agent to reduce the size of a tumor or to prevent the tumor from growing comprises the steps: (1) imaging the tumor with an imaging agent described herein and measuring the size of the tumor; (2) administering the bioactive agent in a sufficient amount to the subject to reduce the size of the tumor or prevent the growth of the tumor; (3) re-imaging the tumor with an imaging agent described herein and measuring the size of the tumor, and (4) comparing the size of tumor after administration of the bioactive agent to the size of the tumor prior to administration of the bioactive agent.

In another aspect, a method for evaluating the ability of a bioactive agent to prevent or reduce plaque growth in one or more blood vessels in a subject comprises the steps: (1) imaging the blood vessels with an imaging agent in any of claims 1-10 and measuring the presence and amount of plaques; (2) administering the bioactive agent in a sufficient amount to the subject to reduce or prevent plaque formation; (3) re-imaging the blood vessels with an imaging agent described herein and measure the presence and amount of plaques, and (4) comparing the amount of plaques after administration of the bioactive agent to the amount of plaques prior to administration of the bioactive agent.

These methods generally involve administering a bioactive agent having a certain therapeutic property and evaluating it performance by comparing a tumor, plaque, or other tissue that can be imaged with the imaging agents described herein prior to and after administration of the bioactive agent.

In addition to accumulating specifically in tumor cells, the imaging agents are readily excreted from the subject. The term “substantially all of the imaging agent is excreted from the subject” is defined herein as the ability not to detect the imaging agent in the blood of the subject by a detection means such as, for example, magnetic resonance after administration to the subject. Not wishing to be bound by theory, it is believed that because the molecular weight of the imaging agent is low it can be readily removed from the blood by, for example, renal filtration. The removal of imaging agent from the subject after examination is another advantage of the present invention.

It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### Examples

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

### Synthesis of CLT1-(Gd-DTPA) Conjugate and Fluorescein-CLT1 Conjugate

CLT1-(Gd-DTPA) (FIG. 2) was synthesized by solid phase peptide synthesis from Fmoc-protected amino acids on a 2-chlorotrityl chloride resin. At the end of the peptide synthesis, an excess of DTPA-dianhydride in DMSO was added to conjugate DTPA at the N-terminal of the peptide. The mixture was shaken in the presence of diisopropylethylamine at room temperature for 6 hours. The resin was completely washed with water, methanol, DCM and DMF 3 to 4 times each. The CLT1-DTPA was then cut from the resin using TFA solution (TFA 94%, 1,2-ethanedithiol 2.5%, Triisobutylsilane 2.5%, water 1%). The solvent was then evaporated under vacuum and the residue was then treated with ether to give a solid product. The solid was exposed to air for about 1 hour to allow the formation of disulfide bonds for the cyclic peptide. The product was purified using preparative HPLC with a C18 column. The targeted contrast agent was prepared by complexation of the ligand with Gd(OAc)₃ at pH 6. Excess Gd(III) was removed by precipitation at pH 11. The final product was further purified by preparative HPLC. CLT1-fluorescein was similarly synthesized using the same procedure as CLT1-(Gd-DTPA).

### Relaxivity Measurement

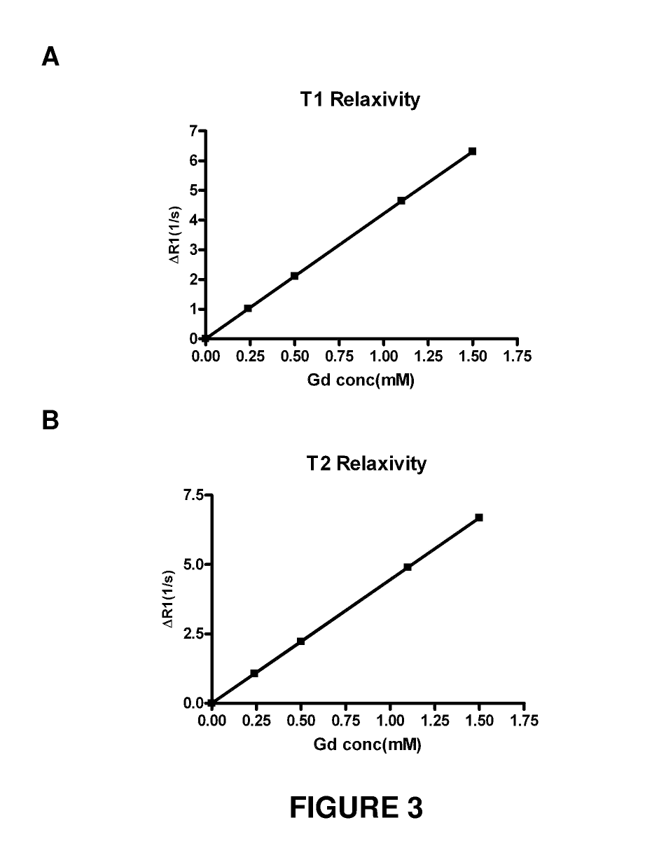

The T₁ relaxation time of water protons in aqueous solutions of CLT1-(Gd-DTPA) with various concentrations was determined on a Siemens Trio 3T scanner at room temperature using an inversion recovery (1R)-prepared turbo spin echo (TSE) imaging pulse sequence using a variety of inversion times (TIs) ranging from 22 ms to 2000 ms. The T₂ relaxation time was measured using a turbo spin echo imaging sequence with turbo factor 3 T₂ values and a serial of echo times TE=12, 24, 36, 47, 59, 71, 83, 95 and 107 ms. The T₁ and T₂ relaxivities (r₁ and r₂) were calculated from the slop of the plot of 1/T_{1,2} vs [Gd] (FIG. 3).

### MR Imaging

The peptide conjugates were evaluated in female athymic nude mice inoculated with two different tumor xenografts, MDA-MB-231 and U87. The animals were cared for under the guidelines of University of Utah Institutional Animal Care and Use Committee. The mice were anesthetized by intramuscular administration of a mixture of ketamine (45 mg/kg) and xylazine (6 mg/kg). A group of 3 mice were used for each tumor model and each contrast agent. The CLT1-(Gd-DTPA) was injected via a tail vein and Gd(DTPA-BMA) was used as a control. For the MDA-MB-231 tumor xenograft model, the dose for CLT1-(Gd-DTPA) and Gd(DTPA-BMA) were 0.05 mmol/kg and 0.1 mmol/kg respectively. The same dose (0.05 mmol/kg) was used for both agents in the U87 tumor xenograft model. Contrast enhanced MR images were acquired before and after injections at 1, 5, 10, 15, 30 and 60 minutes on a Siemens Trio 3T scanner using a human wrist coil. A 3D FLASH sequence with TR 7.75 ms, TE 2.56 ms, slice thickness 0.5 mm, FOV 120 mm, TOF voxel size 0.5×0.5×0.5 mm was used for acquiring high resolution 3D images. Contrast enhanced axial tumor images were also acquired with a T₁-weighted 2D spin echo sequence with TR 7.75 ms, TE 2.56 ms, slice thickness 0.5 mm, FOV 120 mm, TOF voxel size 0.5×0.5×0.5 mm MR images were analyzed using Osirix (Http//:homepage.mac.com/rossetantoine/Osirix/).

### Fluorescence Imaging

CLT1-fluorescein was intravenously injected into mice with MDA-MB-231 xenografts at a dose of 0.01 mmol/kg. The mice were sacrificed at 2 hours after injection and tumor tissues were excised. Tumor tissues from mice without the injection of CLT1-fluorescein were used as negative controls. The fluorescence images of tumor tissues were acquired on an Olympus BX51WI fluorescence microscope equipped with a 100-W high pressure mercury lamp. Images were processed with the use of IMAGE PRO PLUS 3.1 software (Media Cybernetics, Silver Springs, Md.).

### Targeted Contrast Agent CLT1-(Gd-DTPA)

The peptide CLT1 was first synthesized using solid-phase chemistry and DTPA was then conjugated to the peptide by reacting DTPA dianhydride to N-terminal amino group of the peptide on beads. Cyclic peptide DTPA conjugate was formed by autoxidation of cysteinyl residue after it was removed from the beads. The targeted contrast agent was finally prepared by the complexation of Gd(OAc)₃ with the ligand in high yield. The structure of the contrast agent was confirmed by mass spectrometry. FIG. 2 shows the structure of CLT1-(Gd-DTPA) and FIG. 3 shows the plots of the T₁ and T₂ water proton relaxation rates at various concentrations of the contrast agents and 3T. The T₁ and T₂ relaxivities of CLT1-(Gd-DTPA) were 4.22 and 4.45 mM⁻¹sec⁻¹ at 3T.

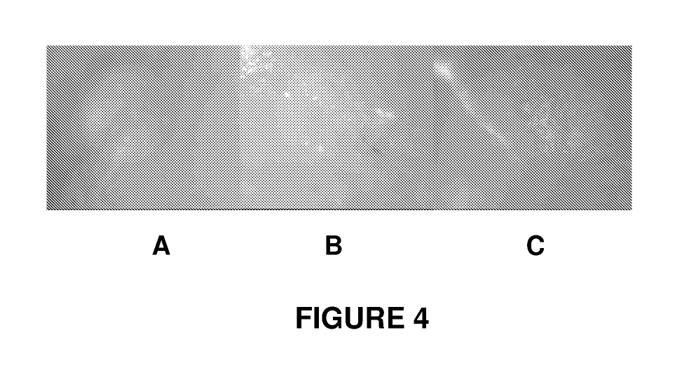

The in vivo tumor binding of CLT1 peptide was verified by tumor fluorescent imaging after injection of a CLT1-FITC conjugate in mice bearing MDA-MB-231 human breast carcinoma xenografts. FIG. 4 shows the fluorescence images of excised tumor tissues from the mice without and with the injection of CLT1-FITC. Strong fluorescence was shown in the tumor tissues from the mice injected with CLT1-FITC while no significant fluorescence was observed in the control tumor. The results confirmed the strong binding affinity of the labeled peptide to tumor tissue after intravenous injection.

### In Vivo MR Molecular Imaging in MDA-MB-231 Xenografts

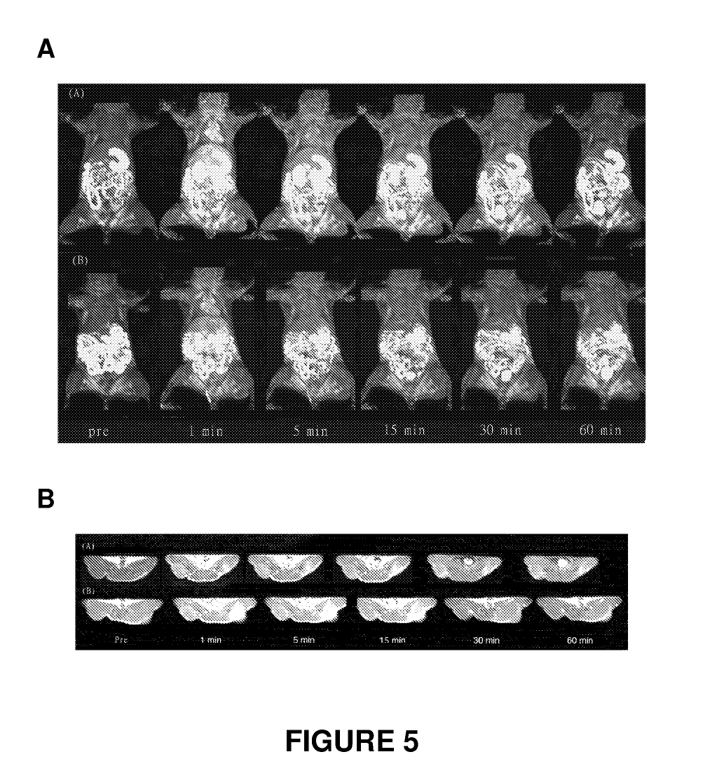

The pharmacokinetics and biodistribution of the targeted contrast agent was non-invasively evaluated in mice bearing MDA-MB-231 human breast carcinoma xenografts with high-resolution three-dimensional dynamic contrast enhanced MRI. FIG. 5A shows the representative 3D maximum intensity projection (MIP) images of mice before and after the injection of contrast agents, and the dynamic signal-to-noise ratios (SNR) in the blood, liver, kidney and muscle in mice.

In vivo molecular imaging of fibrin-fibrinectin complexes in tumor with the targeted contrast agent and MRI was evaluated with a T₁-weighted 2D spin-echo sequence. FIG. 5B shows the axial 2D images and SNR in the tumor tissues of the mice bearing MDA-MB-231 tumor xenografts before and after the injection of the CLT1-(Gd-DTPA) (0.05 mmol/kg) and Gd(DTPA-BMA) (0.1 mmol/kg). Significant tumor enhancement was observed in tumor tissues for both agents in the first 15 minutes post-injection. Gd(DTPA-BMA) was then cleared from tumor and tumor enhancement decreased significantly after 30 minutes post-injection. Strong enhancement was still visible in the tumor tissues even at 60 minutes postinjection for CLT1-(Gd-DTPA), indicating binding of the targeted contrast agent to tumor tissue.

### In Vivo MR Molecular Imaging of U87 Xenografts

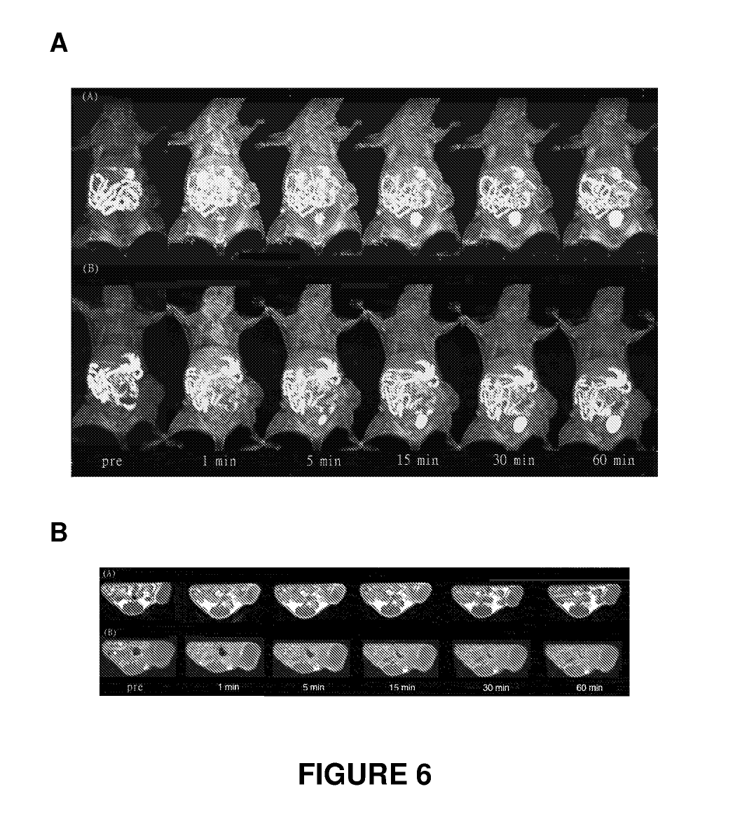

The in vivo properties and specific tumor imaging of CLT1-(Gd-DTPA) were also evaluated in mice bearing U87 human glioblastoma xenografts with Gd(DTPA-BMA) as a control at the same dose. The high-resolution 3D dynamic MRI showed that both agents had similar pharmacokinetics and biodistribution in the mice with U87 xenografts as those in the mice bearing MDA-MB-231 xenografts, FIG. 6A. The T₁ weighted 2D spin-echo images showed that the targeted contrast agent resulted in more significant enhancement in tumor than Gd(DTPA-BMA), FIG. 6B.

### In Vivo MR Molecular Imaging in HT-29 Xenografts

In vivo molecular imaging of fibrin-fibronectin complexes in tumor with the targeted contrast agent and MRI was evaluated in female athymic nu/nu mice bearing HT-29 human colon carcinoma xenografts with a T1-weighted 2D spin-echo sequence. A clinical contrast agent, Gd(DTPA-BMA), was used as a control. Competitive targeting of free CLT1 peptide to CLT1-(Gd-DTPA) was also studied with coinjection of CLT1-(Gd-DTPA) and a 3-fold excess of free CLT1 peptide. A group of 3 mice were used for each contrast agent. The mice were anesthetized by intramuscular administration of a mixture of ketamine (45 mg/kg) and xylazine (6 mg/kg) for MRI. The CLT1-(Gd-DTPA) and Gd(DTPA-BMA) was intravenously injected at a dose of 0.1 mmol/kg. Contrast enhanced MR images were acquired before and after injection at 1, 10, 20, 30 and 60 minutes on a Siemens Trio 3T scanner using a human wrist coil. High resolution 3D images were acquired with a 3D FLASH sequence with 25° flip angel, TR/TE=7.8/2.7 ms, slice thickness 0.5 mm, field of view (FOV) 120 mm, voxel size 0.5×0.5×0.5 mm T1-weighted 2D axial tumor images were acquired with a 2D spin echo sequence with TR 400 ms, TE 10 ms, 90° tip angel and FOV 50 mm Mice were sacrificed 24 hours post-injection and tumor tissues were cut and fixed with 3% paraformaldehyde and embedded in paraffin. Tissue sections (4-μm thickness) were incubated in 3% H₂O₂ for 10 minutes to block endogenous peroxidase activity and boiled in antigen retrieval solution for 15 minutes in microwave. The sections were then incubated with primary antibody overnight and secondary antibody the second day for visualization using the ABC kit (Santa Cruz Biotechnology).

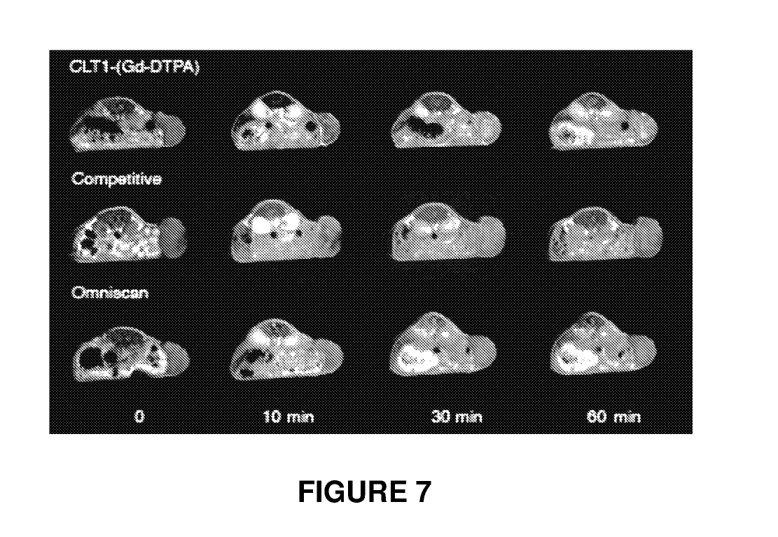

FIG. 7 shows the representative axial T₁-weighted 2D spin-echo images of the tumor tissues of the mice bearing HT-29 tumor xenografts before and after injection of the contrast agents. Significant enhancement was observed in tumor tissues for CLT1-(Gd-DTPA) 10 minutes post-injection, and strong enhancement was visible in the tumor tissues at 60 minutes after injection. For the competitive study, the enhancement of CLT1-(Gd-DTPA) was strongly reduced after the coinjection of free peptide, which indicates that the free peptide recognizes the same binding site within tumor tissue as CLT1-(Gd-DTPA). The control agent Gd(DTPA-BMA) didn't show a strong tumor enhancement comparing with CLT1-(Gd-DTPA), and was cleared out fast 60 minutes post-injection.

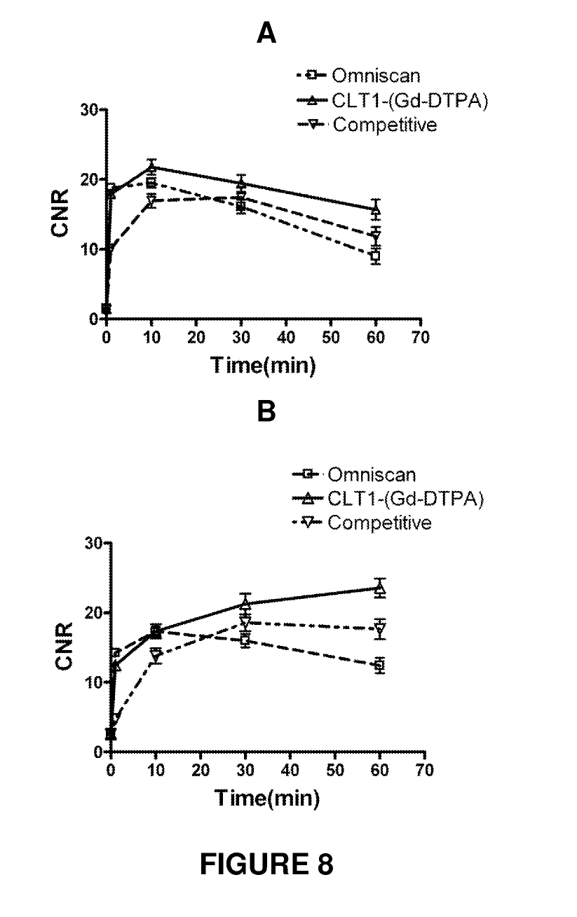

MR signal intensity was also measured and the contrast to noise ratio (CNR) in the tumor tissues was calculated as CNR=(SIₜᵢₛₛᵤₑ−SI_{muscle})/SDₙₒᵢₛₑ. FIG. 8 shows the normalized CNR of the tumor tissue in both the periphery and inner areas before and at various time points after injecting the contrast agents. CLT1-(Gd-DTPA) showed higher CNR at 10 minutes after injection in the tumor periphery area and decreased gradually 60 minutes post-injection. At the same time, CNR in tumor inner area increased gradually during the period of 60 minutes post-injection. The CNR in both tumor periphery and inner areas with Gd(DTPA-BMA) decreased rapidly after it reached the maximum values. The CLT1-(Gd-DTPA) showed significantly higher CNR than the control Gd(DTPA-BMA) in tumor periphery since 10 minute post injection (p<0.05) and in the tumor inner area at 60 minutes post-injection (p<0.05). The results indicate binding and retention of the targeted contrast agent to tumor tissue. The presence of free peptide resulted in significant reduction of CNR in the tumor periphery in first 10 minutes post-injection (p<0.05) and in the tumor periphery since 30 minutes post-injection. The results indicated that the presence of free peptide inhibited the binding of the targeted contrast agent to its target.

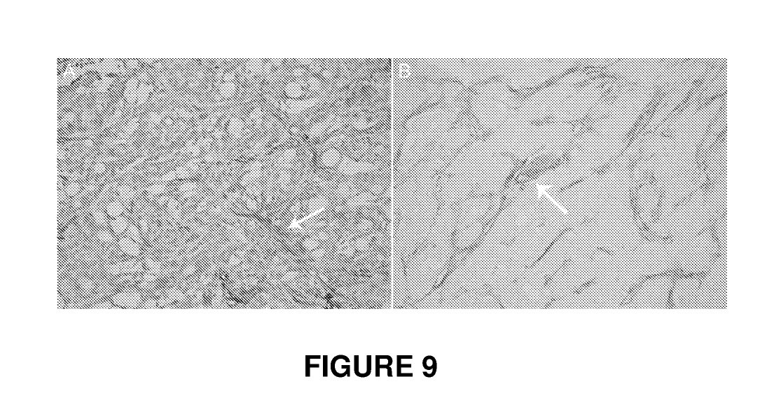

Immunohistology studies were performed in order to verify fibronectin distribution in the HT-29 tumor tissues after MR imaging. FIG. 9 shows the immunostaining of fibronectin in HT-29 tumor tissues with (FIG. 9A) or without (FIG. 9B) nucleus staining. The histochemical staining clearly indicated the existence of fibronectin in the extracellular spaces of tumor tissues.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A compound comprising a cyclic peptide covalently attached to an imaging agent, wherein the cyclic peptide comprises the sequence SEQ ID NO 1 or SEQ ID NO 2, a linear peptide comprising the sequence SEQ ID NO 3, or any degenerate variant thereof.

2. The compound of claim 1, wherein the imaging agent comprises an optical dye, a MRI contrast agent, a PET probe, a SPECT probe, a CT contrast agent, or an ultrasound contrast agent.

3. The compound of claim 1, wherein the imaging agent comprises a chelating agent and a metal ion.

4. The compound of claim 3, wherein the chelating agent comprises ethylenediamine, diethylenetriaminepentaacetate (DTPA) or its derivatives, 1,4,7,10-tetraazadodecanetetraacetate (DOTA) or its derivatives, 1,4,7,10-tetraazadodecane-1,4,7-triacetate (DO3A) or its derivatives, ethylenediaminetetraacetate (EDTA) or its derivatives, 1,4,7,10-tetraazacyclotridecanetetraacetic acid (TRITA) or its derivatives, 1,4,8,11-tetraazacyclotetradecane-1,4,8,11-tetraacetic acid (TETA) or its derivatives, 1,4,7,10-tetraazadodecanetetramethylacetate (DOTMA) or its derivatives, 1,4,7,10-tetraazadodecane-1,4,7-trimethylacetate (D03MA) or its derivatives, N,N′,N″,N′″-tetraphosphonatomethyl-1,4,7,10-tetraazacyclododecane (DOTP) or its derivatives, 1,4,7,10-tetraazacyclododecane-1,4,7,10-tetrakis(methylene methylphosphonic acid) (DOTMP) and its derivatives, MJJO-tetraazacyclododecane-MJJO-tetrakis(methylene phenylphosphonic acid) (DOTPP) or its derivatives, or N,N′-ethylenedi-L-cysteine or its derivatives.

5. The compound of claim 3 wherein the metal ion comprises Gd+3, Eu+3, Tm+3, Dy+3, Yb+3, Mn+2, or Fe+3, 55Co, 64Cu, 67Cu, 47Sc, 66Ga, 68Ga, 90Y, 97Ru, 99 mTc, 111In, 109Pd, 153Sm, 177Lu, 186Re, or 188Re.

6. The compound of claim 1, wherein the imaging agent comprises a PET or SPECT imaging agent, wherein the agent comprises 55Co, 64Cu, 67Cu, 47Sc, 66Ga, 68Ga, 90Y, 97Ru, 99mTc, 111In, 109Pd, 153Sm, 177Lu, 186Re, or 188Re coordinated to a chelating agent

7. The compound of claim 1, wherein the imaging agent comprises an MRI agent, wherein the MRI agent comprises a chelating agent and a metal ion comprising Gd+3, Eu+3, Tm+3, Dy+3, Yb+3, Mn+2, or Fe+3 ions.

8. The compound of claim 1, wherein the imaging agent comprises Gd+3 chelated to diethylenetriaminepentaacetate (DTPA).

9. The compound of claim 1, wherein the imaging agent comprises Gd+3 chelated to diethylenetriaminepentaacetate (DTPA) and the peptide is SEQ ID NO 1.

10. The compound in claim 1, wherein the compound comprises a pharmaceutically acceptable salt or ester thereof.

11. A pharmaceutical composition comprising a compound in claim 1 and a pharmaceutically acceptable carrier.

12. A method for imaging a tissue in a subject comprising (1) administering to the subject an imaging agent in claim 1, and (2) detecting the imaging agent.

13. A method for imaging a tissue in a subject comprising (1) contacting the tissue with an imaging agent in claim 1, and (2) detecting the imaging agent.

14. The method of claim 12, wherein the tissue comprises a tumor, wound or plaque, respectively.

15. The method of claim 14, wherein the cancer comprises breast cancer, liver cancer, stomach cancer, colon cancer, pancreatic cancer, ovarian cancer, lung cancer, kidney cancer, prostate cancer, testicular cancer, glioblastoma, sarcoma, bone cancer, brain cancer, head-and-neck cancers, or skin cancer.

16. The method of claim 13, wherein the contacting step is in vitro, in vivo, or ex vivo.

17. A method for imaging an atherosclerotic tissue in a subject comprising (1) administering to the subject an imaging agent in claim 1, and (2) detecting the imaging agent.

18. A method for imaging an atherosclerotic plaque in a subject comprising (1) administering to the subject an imaging agent in claim 1, and (2) detecting the imaging agent.

19. A method for evaluating the ability of a bioactive agent to reduce the size of a tumor or to prevent the tumor from growing, comprising (1) imaging the tumor with an imaging agent in claim 1 and measuring the size of the tumor; (2) administering the bioactive agent in a sufficient amount to the subject to reduce the size of the tumor or prevent the growth of the tumor; (3) re-imaging the tumor with an imaging agent in claim 1 and measuring the size of the tumor, and (4) comparing the size of tumor after administration of the bioactive agent to the size of the tumor prior to administration of the bioactive agent.

20. A method for evaluating the ability of a bioactive agent to prevent or reduce plaque growth in one or more blood vessels of a subject, comprising (1) imaging the blood vessels with an imaging agent in claim 1 and measuring the presence and amount of plaques; (2) administering the bioactive agent in a sufficient amount to the subject to reduce or prevent plaque formation; (3) re-imaging the blood vessels with an imaging agent in claim 1 and measure the presence and amount of plaques, and (4) comparing the amount of plaques after administration of the bioactive agent to the amount of plaques prior to administration of the bioactive agent.

21. A method for imaging a fibrin-fibronectin complex in a subject comprising
(1) administering to the subject an imaging agent in claim 1, and
(2) detecting the imaging agent.

22. The method of claim 21, wherein the fibrin-fibronectin complex is present in a tumor.

23. The method of claim 21, wherein the fibrin-fibronectin complex is present in a plasma clot.


SEQUENCE LISTING (3 sequences)

SEQ ID NO: 1 - protein, 10 aa, Artificial Sequence
  Cyclic peptide that covalently attaches to an imaging agent
CGLIIQKNEC

SEQ ID NO: 2 - protein, 10 aa, Artificial Sequence
  Cyclic peptide that covalently attaches to an imaging agent
CNAGESSKNC

SEQ ID NO: 3 - protein, 5 aa, Artificial Sequence
  Linear peptide that covalently attaches to an imaging agent
CREKA